(19) United States
(12) Patent Application Publication
(10) Pub. No.: US 2013/0059013 A1
(43) Pub. Date: Mar. 7, 2013

(54) INJECTABLE OSTEOINDUCTIVE BONE CEMENTS

(71) Applicants: Chiara Vitale Brovarone, Torino (IT); Enrica Verne', Torino (IT); Mauro Bergui, Mondovi' (Cuneo) (IT); Barbara Onida, Torino (IT); Francesco Baino, Asti (IT); Marta Miola, Torino (IT); Sara Ferraris, Grugliasco (Torino) (IT); Francesca Tallia, Biella (IT)

(72) Inventors: Chiara Vitale Brovarone, Torino (IT); Enrica Verne', Torino (IT); Mauro Bergui, Mondovi' (Cuneo) (IT); Barbara Onida, Torino (IT); Francesco Baino, Asti (IT); Marta Miola, Torino (IT); Sara Ferraris, Grugliasco (Torino) (IT); Francesca Tallia, Biella (IT)

(73) Assignee: POLITECNICO DI TORINO, Torino (IT)

(21) Appl. No.: 13/697,076

(22) PCT Filed: May 12, 2011
(86) PCT No.: PCT/IB11/52094

§ 371 Date: Nov. 9, 2012

(30) Foreign Application Priority Data
May 13, 2010 (IT) TO2010A000401

(51) Int. Cl. A61K 33/42; A61P 19/08

(52) U.S. Cl. 424/602

(57) ABSTRACT

An injectable composition for bone filling or consolidation, for use in surgery or therapy, characterized in that it comprises a biocompatible powdery material, which is hardenable or cross-linkable and selected from calcium sulphate hemi-hydrate and polymethylmethacrylate, a hardening or cross-linking agent for said material and a porous silica-based powdery material, having a specific surface area higher than 100 m²/g, which comprises a mesoporous bioactive glass, obtainable/achievable by a sol-gel method with the addition of a surfactant as structure-directing agent for pores formation, comprising an oxide mixture containing:

SiO₂: from 60 to 85% by moles
CaO: from 10 to 30% by moles
P₂O₅: from 2 to 10% by moles
said composition, when applied to an affected area of a patient, being adapted to form a composite material wherein said hardenable or cross-linkable material constitutes a matrix and said mesoporous bioactive glass constitutes a dispersed phase in said matrix.

The present invention concerns an injectable composition for the use in bone-filling and bone-consolidation in surgery and therapy.

In particular, the invention relates to the field of injectable bone cements, for both treating of fractures caused by osteoporosis or trauma and filling gaps due, for example, to the decrease of bone mass after removal of tumors or cysts.

Furthermore, this invention may be useful to strengthen the bone structure with a view to the subsequent implant of screws or prostheses.

The request of such materials is ever growing because of the increased life expectancy.

Bone cements are generally prepared in the form of pastes that can be injected directly into the fracture site, so that, after hardening, they fill the vacuum (or consolidate the fracture) and support the surrounding bone. It is clear that such bone substitutes must provide a good short-term stability and prevent micro-movements that can delay or inhibit tissue regeneration: thus, the injected material has to harden quickly and to get fixed with the surrounding bone tissue.

The present invention is related to injectable materials that can be used in various pathological or traumatic conditions in maxillo-facial and orthopedic surgery, as well as in different applications of neurosurgery, such as vertebroplasty and kyphoplasty.

These two spinal surgical techniques are used for the treatment of osteoporotic vertebral fractures, traumatic fractures and in case of particular spinal tumors, in order to stabilize as much as possible the weakened vertebral body, to relieve the pain due to the fracture, especially when it becomes high and persistent; in addition, kyphoplasty aims also to restore the height of the vertebra to as near its pre-fracture physiological level as possible and to correct possible kyphotic deformities caused by the fracture.

These surgical approaches are increasingly frequent because, without them, the pain caused by a spinal fracture often requires a more or less prolonged bed rest. Patients in good general conditions tolerate this conservative treatment without excessive problems and, in most cases, they heal with a complete resolution of symptoms and a quick recover of their autonomy.

Instead, bed rest represents a significant risk factor for “critical” patients, because of the possible associated severe co-morbidity: this population includes patients with limited prognosis (“very old” people, patients affected by not-controlled solid tumors), patients with a potential good prognosis after overcoming the acute phase (patients with severe liver disease awaiting transplant or in the immediate post-transplant, haematological patients receiving chemotherapy or bone marrow transplant). In these cases the percutaneous stabilization provided by vertebroplasty or kyphoplasty allows a rapid recovery of standing and walking abilities, avoiding the co-morbidity related to both the bed rest and the use of more painkillers.

These procedures usually consist in the percutaneous injection of an acrylic bone cement (based on polymethylmethacrylate—PMMA) directly into the fractured vertebral body (in case of vertebroplasty) or into a cavity created inside the vertebral body through the inflation of a balloon (in case of kyphoplasty).

After the injection, the cement hardens through polymerization and/or crosslinking processes, giving the patient an immediate sense of relief because of the decrease or even elimination of pain due to the stabilization of the vertebral body.

After injection and setting, PMMA is gradually included by a capsule of fibrous scar tissue, that makes the cement fixed with surrounding bone tissue, but it is completely inert, so it will be neither reabsorbed nor osteointegrated. Thus, the mechanical properties of the vertebral body containing PMMA are different from those of the adjacent vertebrae, resulting in possible consequences that could affect the stability of the surrounding bone (in case of vertebroplasty/kyphoplasty the risk of secondary fractures in the vertebrae adjacent to the treated one is well documented in scientific literature).

The complications are mainly related to possible cement leakage outside the vertebral body, localized temperature rise produced by hardening of acrylic cements, and, especially in case of seriously immunocompromised patients, infections.

The most desirable properties that these type of cements should possess are easy injectability, radio-opacity, mechanical properties of the hardened cement that are comparable to those of a healthy intact vertebral body.

Ideally, especially for patients with long life expectancy, the hardened cement should be gradually bioresorbed during in vivo osteointegration phenomena, with a resorption rate comparable to the kinetics of healthy bone tissue regeneration.

In fact, cements based on bioinert materials represent a permanent implant which is, therefore, barely indicated for the treatment of patients with long life expectancy.

To this end, calcium sulphate hemi-hydrate (commonly known as “Plaster of Paris”), CaSO₄.0.5H₂O, was one of the first materials investigated for bone replacements and its high biocompatibility as well as its ability to be bioresorbed, are widely documented in literature.

US Patent No. 2009/0068272 A1 describes the procedure for the synthesis of mesoporous calcium silicate powders for controlled release of bioactive agents. Paragraphs 0024 and 0031 describe particles, whose dimension ranges between 20 nm and 75 nm, obtained through acid treatment on wollastonite powders, that causes the formation of surface mesoporosity. The size of nanoparticles and the average diameter of mesopores are determined by the conditions selected for the acid treatment. Experimental verification demonstrates that the above-mentioned mesoporous calcium silicate powders can be neither mixed nor injected in combination with a proper matrix (for example, matrices in calcium sulphate hemi-hydrate or polymethylmethacrylate), resulting thus unsuitable for the preparation of injectable bone cements.

One object of the present invention is to provide a novel injectable composition, helpful for the above-mentioned purposes, in particular a composition which would result in a rapid bone regeneration.

A further object of the invention is to provide a composition which can be used as vehicle to carry biologically active substances directly to the injection site.

For such purposes, object of the invention is an injectable composition having the characteristics defined in the claims that follow, as well as the resulting bone cement.

The composition according to the invention, when applied to an affected area of a patient, forms a composite bone cement which comprises a first phase, representing the matrix, and at least one dispersed phase.

The phase acting as the matrix may be constituted by a biocompatible powder that is able to generate, through the contact with a liquid, a solution from which one or more crystalline phases precipitate. Alternatively, the matrix may be constituted by a biocompatible powder material that, when placed in contact with a proper polymerizable liquid, is able to generate a solid material, which is mechanically resistant.

The matrix material can be both organic or inorganic; the preferred material is calcium sulphate hemi-hydrate, preferably α-calcium sulphate hemi-hydrate, which possibly may include particles of calcium sulphate di-hydrate in order to improve the injectability of the composition. Calcium sulphate hemi-hydrate is converted into calcium sulphate di-hydrate through the reaction with water, making the mixture supersaturated with respect to calcium sulphate di-hydrate, which precipitates as needle-like crystals, whose entanglement causes firstly the setting and then the hardening of the injected material.

A matrix based on calcium sulphate is completely bioresorbable in contact with body fluids in an average period of time of just over a month.

In addition, inorganic matrix materials may include mixtures of different calcium phosphates, that are able to generate hydrated phases through the contact with aqueous solutions, or mixtures of calcium phosphates and calcium sulphate hemi-hydrate.

Not bioresorbable organic matrices in polymethylmethacrylate in presence of the corresponding activator agent (for example, benzoyl peroxide) are also envisaged.

The injectable composition object of the invention also includes at least a second phase that, in the resulting composite bone cement, acts as dispersed phase. This dispersed phase comprises a silica-based material, which contains other oxides, such as CaO and P₂O₅, and optionally ZrO₂ and SrO, and which is characterized by an high specific surface area, greater than 100 m²/g.

Preferred dispersed phases are porous materials, with a pores size variable within the range of mesopores, that is typically from 2 nm to 50 nm, particularly bioactive glasses with controlled mesoporosity.

Bioactive glasses with controlled mesoporosity represent a special class of nanomaterials, i.e. materials whose properties can be tuned at the nanoscale, whose common specific feature is the presence of uniform nanopores whose size ranges within 2÷50 nm.

The synthesis of bioactive glasses with controlled mesoporosity is based on the sol-gel method, which has the advantage of being not expensive and simple and that is used to realize ceramic materials starting from liquid precursors. In the sol-gel process, which is a technique generally used for the fabrication of glass or ceramic materials, a liquid colloidal solution, commonly known as “sol”, that is constituted by a suspension of solid particles (usually metal hydroxides and alkoxides or inorganic salts) into a liquid phase, gradually evolves, through hydrolysis and polycondensation reactions, towards the formation of a gel; this gel is, then, thermally treated to produce high-purity oxides. In the synthesis of bioactive glasses with controlled mesoporosity, this sol-gel method is coupled with a supermolecular self-assembly process of proper amphiphilic molecules, consisting in an organic surfactant. Specifically, the surfactant is introduced into the traditional sol-gel synthesis of bioactive glasses by its addition directly in the solution that forms the “sol”, enabling to exploit the contemporary properties of hydrophilicity (tendency to dissolve in polar solvents) and hydrophobicity (tendency to dissolve in apolar environments) of such surfactant. As a result of their amphiphilic nature, surfactant molecules are able to form, through a self-assembly process, supermolecular aggregates, named micellae, whose shape and spatial arrangement can be controlled on the basis of the selection of type and concentration of surfactant.

The latter, in particular, is removed through a calcination thermal treatment, leading to a bioactive glass material with controlled mesoporosity (MBG) characterized by an extremely high accessible surface area (higher than 100 m²/g and generally of the order of 300-500 m²/g). This feature, in combination with the effective control of size, distribution and order of the pores, makes it possible to act selectively controlling and tuning the properties and the behaviour to be attributed to the resulting mesoporous material, based on the type of chosen surfactant. As an example, FIG. 14 reports the spectrum resulted from X-ray diffraction analysis on MBG powders: diffraction peaks clearly visible within the angular range 1°-2° represent an experimental evidence of the ordered structure typical of the above-described MBGs, whose nanopores are both uniform and with controlled size.

Numerous surfactants, both ionic and non-ionic, can be used for MBG synthesis, among which the most common are for example: block copolymers of poly(ethylene glycol)poly(propylene glycol)-poly(ethylene glycol) (e.g. Pluronic P123 and F127), cetyltrimethylammonium bromide (CTMABr), isononylphenoxy-poly(ethylene) oxide (NONFIX 10).

Therefore, the presence of the nanoporous structure inside a bioactive glass increases its exposed surface area, accelerating the complex mechanism of ion exchange with body fluids, which is the base of the bioactivity of these materials (e.g. their ability to establish a strong chemical bond with surrounding bone tissue through the rapid formation of submicrometrical hydroxyapatite crystals on their surface). Hence, the in vivo implant of a MBG is potentially able to stimulate a rapid bone regeneration, enabling a more effective and active patient rehabilitation, which represents a very important feature in case of osteoporotic patients.

Moreover, a further advantage consists in the possibility of using MBG nanopores as vehicles for the transport and the controlled release of drugs or other biomolecules in order to relieve pain of patients, reduce inflammation or treat specific diseases. In particular, specific growth factors or anti-tumoral drugs can be transported.

MBGs, according to the present invention, include bioactive glasses with the following composition:

SiO₂: from 60 to 85% by moles;
CaO: from 10 to 30% by moles;
P₂O₅: from 2 to 10% by moles.
and optionally other oxides, such as ZrO₂ e SrO, from 2% to 15% referred to 100% by moles of the 3 above-mentioned oxides.

Preferred specific surface area: 300-500 m²/g (data calculated from nitrogen sorption curve through BET analysis)

Preferred/preferable pores size: 2-10 nm.

According to a preferred but not binding embodiment, MBG, to which examples from 1 to 7 refer, exhibits a unimodal distribution of pores diameters characterized by a maximum peak value of pores size around 4.7 nm, as shown in FIG. 19.

An essential requirement for vertebral cements consists in the need to have a suitable radio-opacity degree to ensure its visualization under radioscopic guidance, both during the positioning inside the vertebral body in the percutaneous injection procedure (in order to observe any possible leakage) and during the subsequent follow-up checks on the patient.

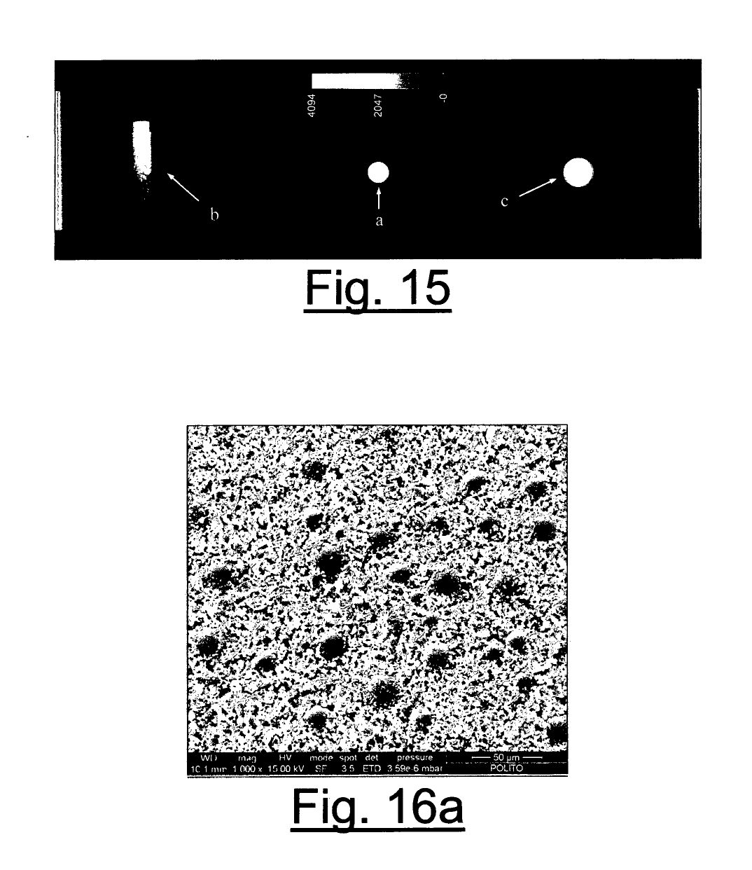

For this reason, in order to impart radio-opacity to the injectable composite material, radioopaque powders, such as BaSO₄, are usually added. In addition, radio-opacity can be achieved using dispersed phases which contain also ZrO₂ and/or SrO₂. In a preferred and innovative embodiment, the invention allows the use of bioactive mesoporous glasses with controlled mesoporosity (MBG) within the range 2-50 nm, preferably from 2 nm to 10 nm, containing an inherently radio-opaque oxide (such as ZrO₂ or SrO), named hereafter MBGZ; in this embodiment the invention provides composite cements that combine bioactive properties and radio-opacity in only one dispersed phase, thus avoiding the need of adding a radio-opaque agent as described above: in fact, the added radio-opaque agent would represent a further dispersed phase which would cause the worsening of miscibility, injectability and mechanical properties, especially bending strength and dynamic fatigue performance, of the composite cement. In particular, as described in detail in examples 8 and 9 reported below, the feasibility of both MBGZ synthesis and introduction of MBGZ particles as dispersed phase within a calcium sulphate matrix has been demonstrated: as shown in FIG. 15, both MBGZ powder and the resulting composite cement are considerably radio-opaque. This fact represents a great innovation of this invention because no publications regarding MBGZ were detected in scientific literature or in patent databases and there is not any composite cement for vertebroplasty that does not contain BaSO₄ or other radio-opaque additives currently available on the market.

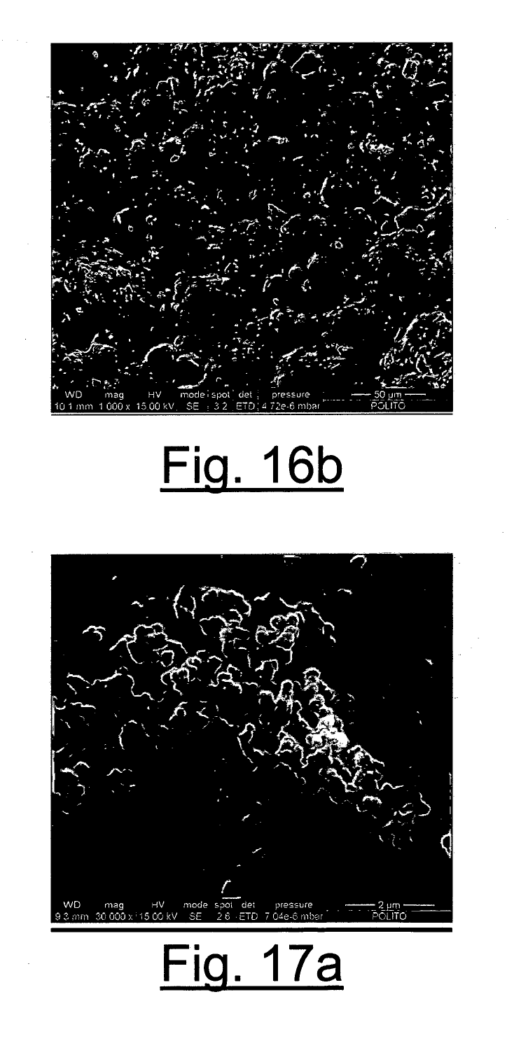

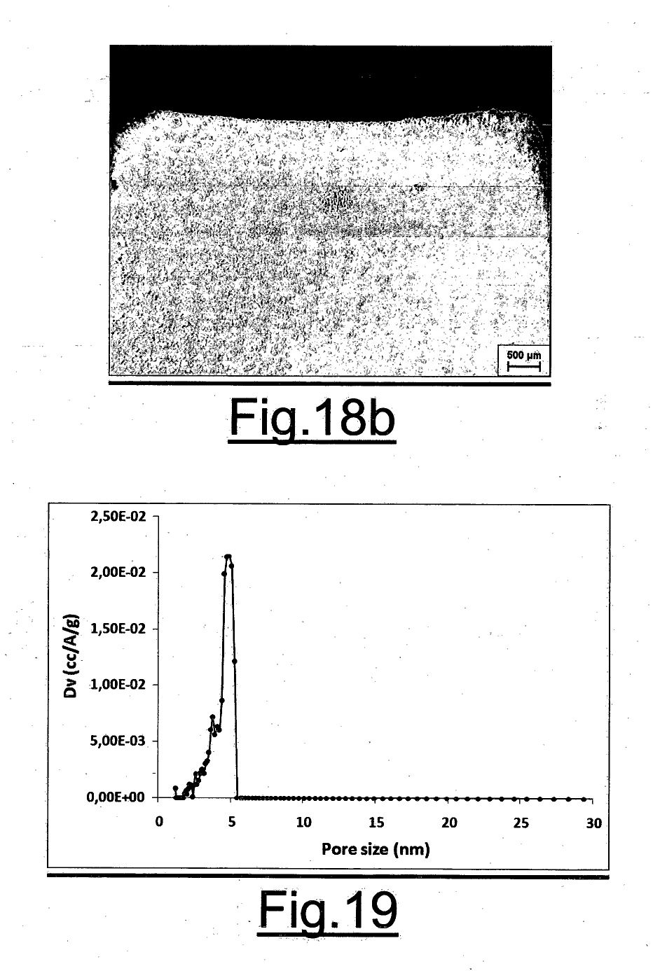

The above-mentioned MBGs are characterized by an osteoinductive behaviour, that consists in an extremely high ability to stimulate the nucleation of sub-micronic crystals of hydroxyapatite on their surface. To this end, preferred size of MBG particles ranges between 5 μm and 32 μm (minimum dimension resulting from sieving). In fact, unexpected experimental evidences have demonstrated that the utilization of a powder granulometry below 32 μm maximizes the exposure of MBG particles on the surface of the resulting hardened cement, since larger particles have difficulty to emerge from the matrix within they are trapped (this effect is clearly visible comparing FIGS. 16a and 16b). MBG exposure is extremely important, because, with a view to the application of the injectable compositions object of the present invention as bone cements, particles exposed on the surface are those which enable the bioactive and osteoinductive effect after the contact with body fluids, feature that is the main purpose of their use as dispersed phase. In this regard, capillarity tests were carried out to evaluate the capillary absorption ability of fluids from hardened composite cements with polymethylmethacrylate matrix, by varying the dispersed phase: in particular, composite cements prepared according to example 6 were compared to cements characterized by the same matrix and the same composition but containing a different dispersed phase, since the MBG powder was substituted by particles of a traditional not-mesoporous bioactive glass with the same size (sieved below 32 μm). Capillarity tests were carried out by partially soaking each sample in a mixture of simulated body fluid (Kokubo's SBF (Kokubo, Takadama—Biomaterials 27 (2006) 2907-2915)) and a coloured liquid, which acts as contrast agent that enables to detect the depth of capillary absorbed fluid from the contact surface. As shown by the observations of the sections of such samples through optical microscope (FIGS. 18a and 18b), a considerable absorption was assessed for the composite containing MBG, since a capillary depth of 600 μm was measured for this sample, whereas no absorption layer was noticed in the composite containing the traditional not-mesoporous bioactive glass. This outcome represents a further unexpected demonstration of the greater technical effect caused by the utilization of MBG with optimized size because particles sieved below 32 μm are highly exposed on the surface and, when the hardened cement is placed in contact with body fluids, their nanopores determine a remarkable capillary absorption that supports and promotes their osteoinductive function, resulting in a considerable increase of MBG bioactivity in respect to a not mesoporosus bioactive glass. Moreover, particles with size lower than 5 μm show a high tendency to agglomerate: this effect irreparably affects the miscibility between solid and liquid phases and the consequent injectability of the resulting paste, preventing therefore to satisfy the viscosity requirements needed for an injectable bone cement.

These glasses may be used as a vehicle for the transport of biologically active substances, including drugs, such as antibiotics, anti-inflammatories, painkillers and anti-tumoral agents, as well as growth factors, such as for example morphogenetic proteins.

In one embodiment, a further phase may be added to the matrix material of the injectable composition, especially in case of bioresorbable injectable matrices: this further material acts as a dispersed phase in the hardened composition, in order to impart better mechanical properties and to provide support and stabilization to the re-growing physiological tissue.

This dispersed phase can be in the form of bioresorbable glass powders or fibres, preferably non-porous, having the following composition:

P₂O₅: from 40 to 60% by moles
CaO: from 10 to 40% by moles (preferably between 20 and 30% by moles)
and optionally containing other metal oxides, such as Na₂O, MgO, K₂O and TiO₂, as a complement to 100% by moles.

The further addition of the above-mentioned bioresorbable dispersed phase is useful to induce, during the osteointegration of the injected material, the formation of a porous network that may be helpful for the transport of nutrients and waste products and can also promote the bone regeneration process.

In order to considerably increase the mechanical properties, especially in case of bioresorbable matrices, another glass material can be introduced as a further dispersed phase in the form of powders or fibres, preferably non-porous, whose size ranges preferably within 5 and 100 μm, having the following composition:

SiO₂: from 40 to 65% by moles
CaO: from 10 to 35% by moles;
and optionally containing other oxides, such as Na₂O, MgO, K₂O, Al₂O₃, ZrO₂ and TiO₂ as a complement to 100% by moles. This dispersed phase can be thermal treated before its addition to the powders, in order to induce a partial devetrification of the glass, resulting in the nucleation of one or more crystalline phases that increase its mechanical strength (glass-ceramic).

In compositions based on a bioresorbable matrix, such as for example calcium sulphate hemi-hydrate, the addition of one or more dispersed phases has also the advantage of reducing the resorption rate of the matrix, making it more compatible with the kinetics of bone tissue regeneration. For example, tests on different cements, composed of simple calcium sulphate di-hydrate or composite materials made by the combination of a calcium sulphate di-hydrate matrix and one or more dispersed phases, were carried out in vitro by soaking the samples in an acellular simulated body fluid that aims to mimic the ion concentration of human plasma (Kokubo's SBF), obtaining the following results:
a cement constituted only by calcium sulphate di-hydrate matrix shows a weight loss of about 25% by weight after 1 week of soaking in SBF;
a composite material, prepared in accordance with example 1 that follows, shows a weight loss of about 8% by weight after 7 days of soaking in SBF;
a composite material, prepared in accordance with example 2 that follows, shows a weight loss of about 7% by weight after 7 days of soaking in SBF;
a composite material, prepared in accordance with example 3 that follows, shows a weight loss of about 5% by weight after 7 days of soaking in SBF.

The percentage of the matrix component is typically from 40% to 95% by weight, referred to the 100% by weight of total amount of matrix and dispersed phase/phases. In particular, the percentage of the matrix material ranges preferably between 60% and 90% by weight.

In case of inorganic matrices for the injectable composition object of the invention, the dry powder, constituted by the matrix and the dispersed phases, is mixed with an aqueous liquid, which also aims to ensure the injectability of the composite paste. This liquid phase can be enriched with a radio-opaque agent and optionally with an oily component that facilitates the injection of the composition.

Typically, the aqueous phase is distilled water or a balanced saline solution; the amount of the aqueous liquid mixed with the powders typically ranges from 0.10 to 1 ml per gram of the total dry powder and preferably between 0.45 and 0.60 ml/g.

In case of organic matrices for the injectable composition object of the invention, the dry powder, constituted by the matrix and the dispersed phases, is mixed with a liquid mixture of monomer, catalyst and inhibitor, that aim at ensuring the injectability and the subsequent hardening of the composite. For example, when a PMMA matrix is used, the monomer is typically MMA (methyl-methacrylate), the catalyst may be dimethyl toluidine, the inhibitor may be hydroquinone.

In this case, the volume of the liquid phase mixed with the powders ranges from 0.20 to 0.80 ml per gram of the total dry powder and preferably between 0.30 and 0.60 ml/g.

Whatever is the material used as matrix, the liquid phase can be enriched with a radioopaque agent and optionally with an oily component that facilitates the injection of the composition.

The viscosity of the injectable material should be adapted to make it injectable into the bone for 1-10 minutes after the beginning of the mixing procedure of the different components. Moreover, the specific formulation of both the composition and the selected liquid phase should be tuned in order to obtain preferably a setting time that varies between 10 and 30 minutes from the dough injection. To achieve the injectability and the setting times described above, a retardant agent, such as for instance a carboxylic acid (e.g. citric acid, succinic acid, malic acid, tartaric acid), can be added either to the powder resulting from the mixture of matrix and dispersed phases (which represents the composition object of the invention) or to the liquid needed to provide the injectability.

The composition can be marketed in the form of a kit that contains measured amounts of each component—matrix material, dispersed phases, liquid phase—as well as all the accessories that the user needs for the preparation and the injection. Further features and advantages of the composition according to the invention will be apparent from the following examples, that, however, should not be construed as limiting the invention in any way.

In the appended drawings:

FIG. 1 illustrates a micrograph of an hydroxyapatite layer precipitated on MBG powders after 24 hours of soaking in SBF;

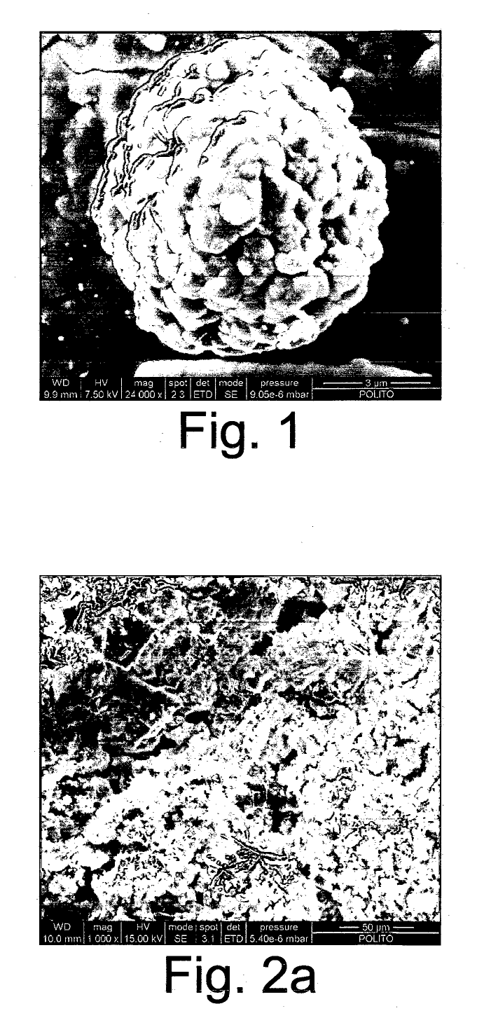

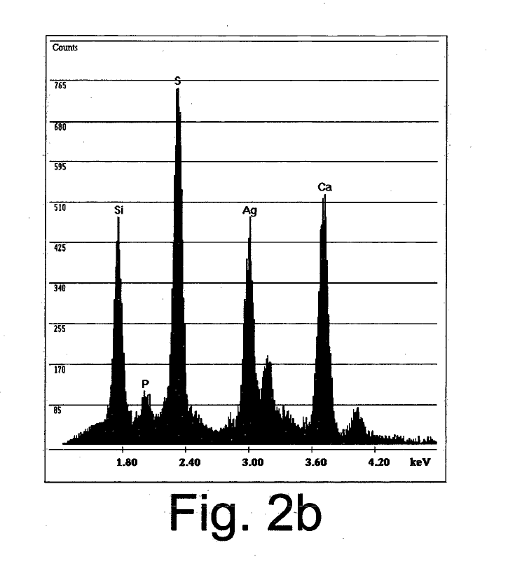

FIG. 2a illustrates a micrograph of the composite material, prepared according to example 1 that follows, constituted by a calcium sulphate hemi-hydrate matrix containing MBG, after 8 hours of soaking in SBF: small agglomerates of hydroxyapatite are visible on the surface of the composite material;

FIG. 2b reports an EDS composition analysis of the whole area exhibited in FIG. 2a: the visible peaks correspond to the constitutive elements of the composite and show an increase in the amounts of Ca and P if compared with those of the starting composition of the dispersed MBG particles, suggesting the precipitation of hydroxyapatite crystals, that demonstrates the extremely high bioactivity of such material;

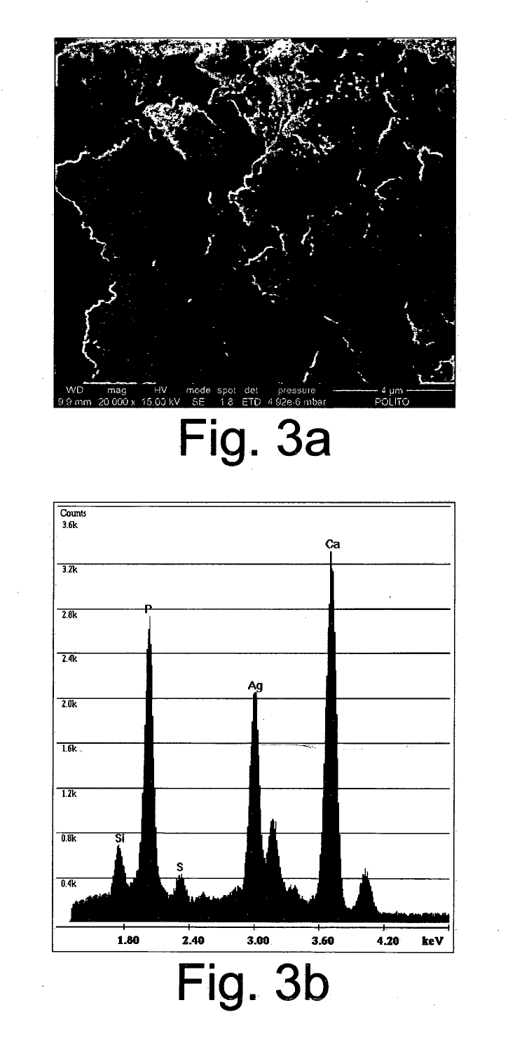

FIG. 3a illustrates a high-magnification micrograph of some agglomerates of hydroxyapatite particles nucleated on the composite prepared according to example 1 after 1 day of soaking in SBF;

FIG. 3b reports an EDS composition analysis of the material surface visible in FIG. 3a, in which the peaks corresponding to Ca and P stand out clearly, confirming that the crystals are actually hydroxyapatite;

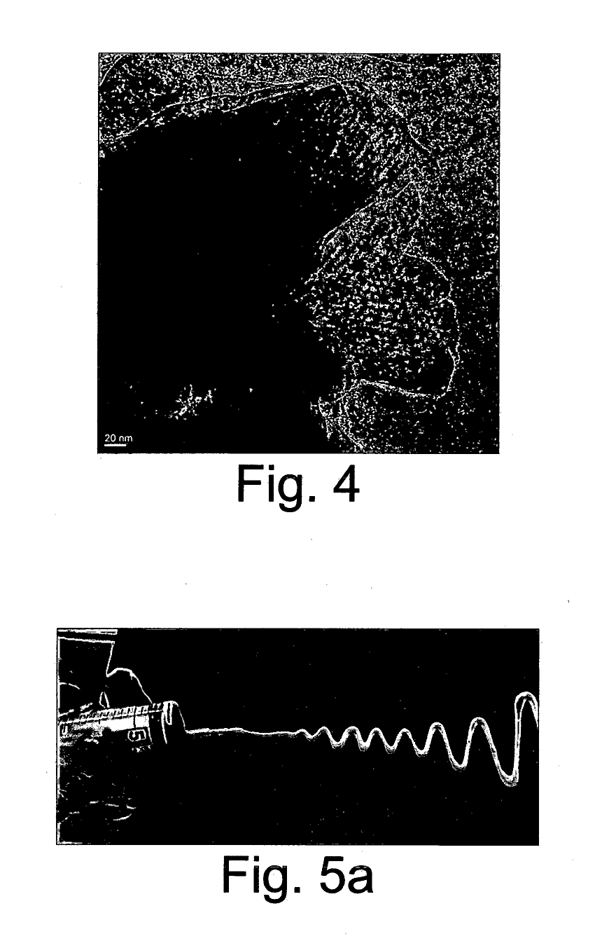

FIG. 4 illustrates a TEM image of the MBG used in all examples from 1 to 7 described below, that shows an ordered and regular nanoporosity;

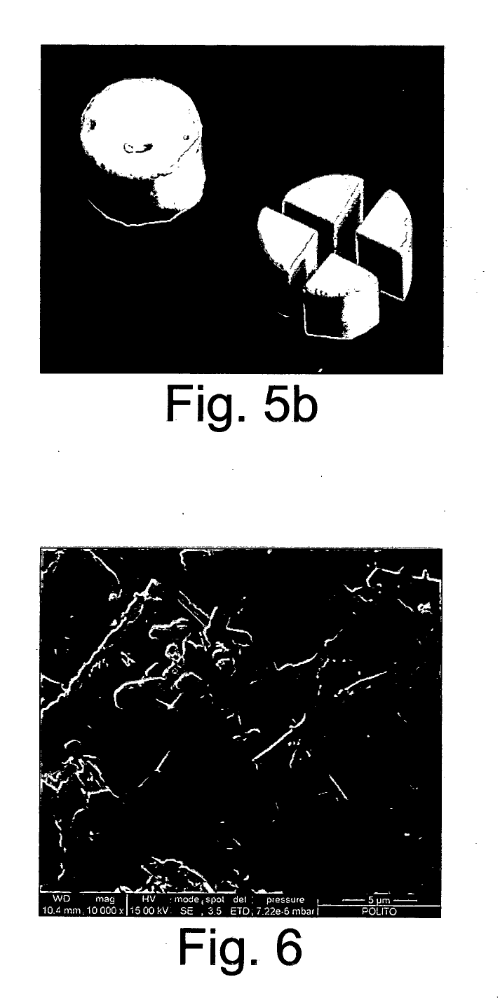

FIG. 5a is a macrograph that illustrates the injectability of the composite material prepared in accordance with examples 2-3 and 4;

FIG. 5b illustrates a macrograph that shows an example of cylindrical sample (diameter=16 mm; height=10 mm) of the composite material obtained according to examples 2-3 and 4, both entire and in section;

FIG. 6 illustrates a micrograph of the surface of the composite material prepared in accordance with example 2, that clearly exhibits calcium sulphate di-hydrate crystals (precipitated after mixing the calcium sulphate hemi-hydrate powder with the liquid phase) among which the particles of the two dispersed phases (MBG and a SiO₂—CaO—Na₂O—Al₂O₃ based glass) are trapped and uniformly distributed;

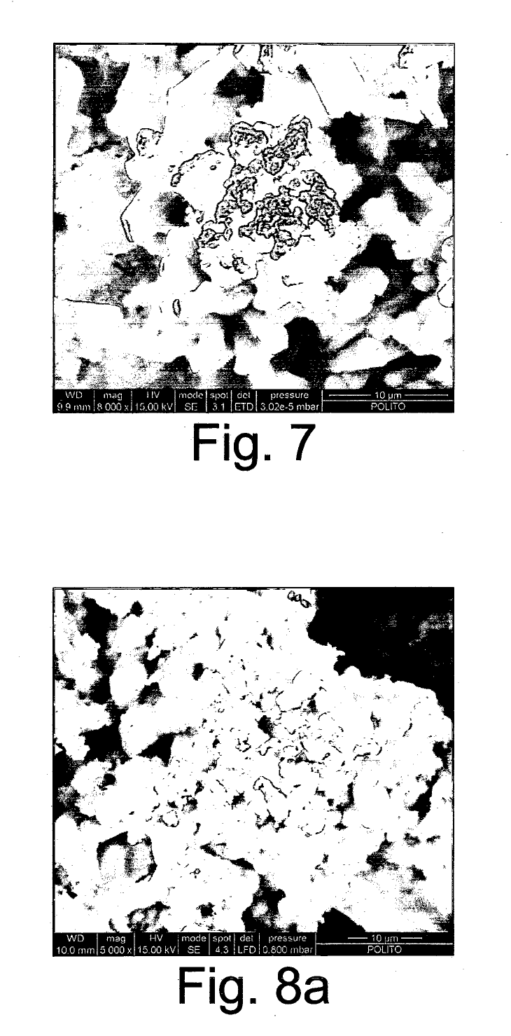

FIG. 7 illustrates a micrograph of the surface of the composite material realized according to example 2 after 1 week of soaking in SBF. In particular, it shows the magnification of some agglomerates of hydroxyapatite particles nucleated on the composite surface: it is important to underline that, despite the excellent bioactive properties of the MBG used as dispersed phase in the composite, the material is not covered by a uniform hydroxyapatite (HAp) layer, because the HAp precipitates are bioeroded with the matrix;

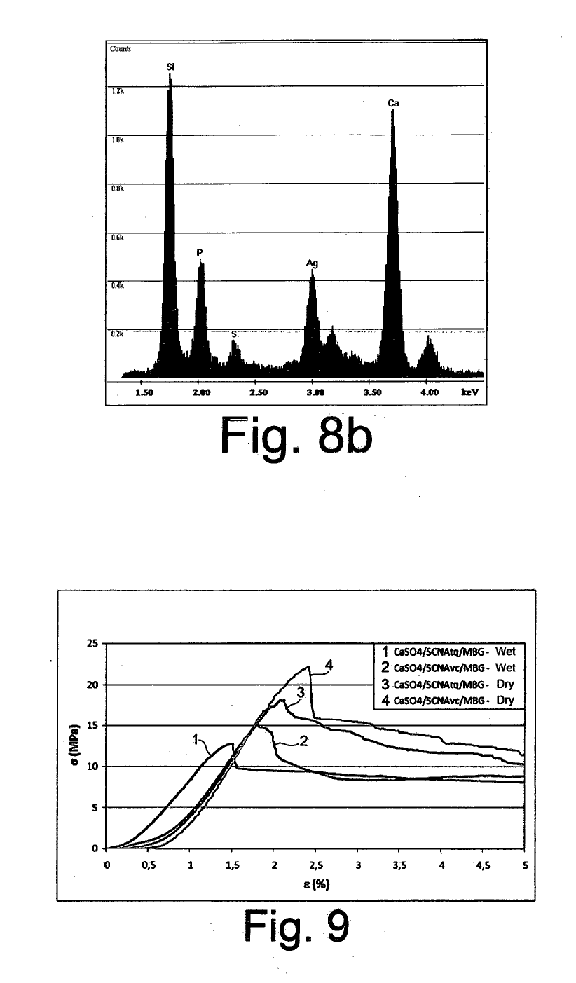

FIG. 8a illustrates a micrograph that shows a magnification of the residual powder filtered from the SBF in which the sample observed in FIG. 7 was soaked: consistently with what is described above, it clearly exhibits a large amount of agglome-rates of hydroxyapatite particles detached from the sample surface because of the resorption;

FIG. 8b reports an EDS composition analysis of a limited area of the material observed in FIG. 8a, in which the height of the peaks corresponding to Ca and P confirms that the micro-crystals are actually hydroxyapatite precipitated on MBG particles, whose presence is proven by the peak of Si; in addition, the peak of S denotes the presence of calcium sulphate di-hydrate;

FIG. 9 is a diagram that illustrates the comparison between the σ-ε curves resulted from the compressive strength testing of the two different composite materials prepared by mixing the calcium sulphate hemi-hydrate powder with MBG particles and a second dispersed phase, in accordance with example 2 (reported as “CaSO₄/SCNAtq/MBG”, in which the second phase is a completely amorphous glass) and example 3 (reported as “CaSO₄/SCNAvc/MBG”, in which the second phase is a glass-ceramic material). Both composites were tested in “wet” conditions (after 24 hours after the preparation), which simulate the in vivo contact with body fluids, and in “dry” conditions (after 1 week after the preparation), leading to the following results in terms of compressive strength:

CaSO₄/SCNAtq/MBG—Wet: 12.7 MPa;

CaSO₄/SCNAvc/MBG—Wet: 15.1 MPa;

CaSO₄/SCNAtq/MBG—Dry: 17.0 MPa;

CaSO₄/SCNAvc/MBG—Dry: 22.1 MPa.

Consistently with the theoretical expectations, the presence of the second dispersed phase in form of a glass-ceramic material provides an increase in the mechanical properties of the composite in which it is introduced, if compared with the same material characterized by the second dispersed phase in form of a completely amorphous glass. Moreover, an increase in both the strength and the stiffness of the above-mentioned composite materials is observed after the complete evaporation of the excess of water, needed to ensure their injectability;

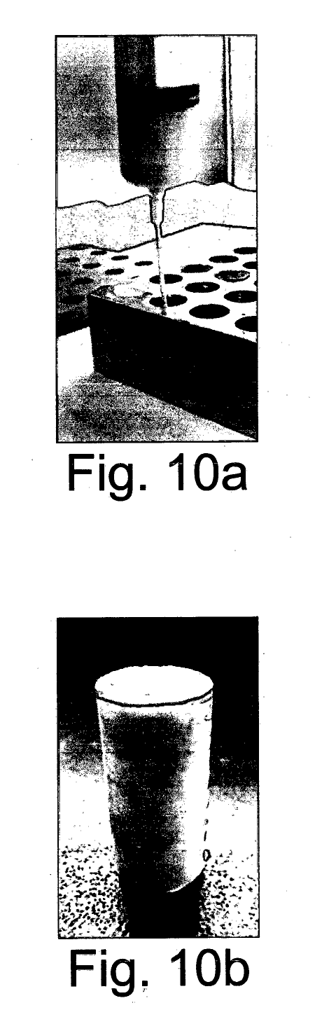

FIG. 10a illustrates a macrograph that demonstrates the injectability of the composite material prepared in accordance with examples 5-6 and 7;

FIG. 10b illustrates a macrograph that shows an example of cylindrical sample (diameter=12 mm; height=24 mm) of the composite material obtained according to examples 5-6 and 7;

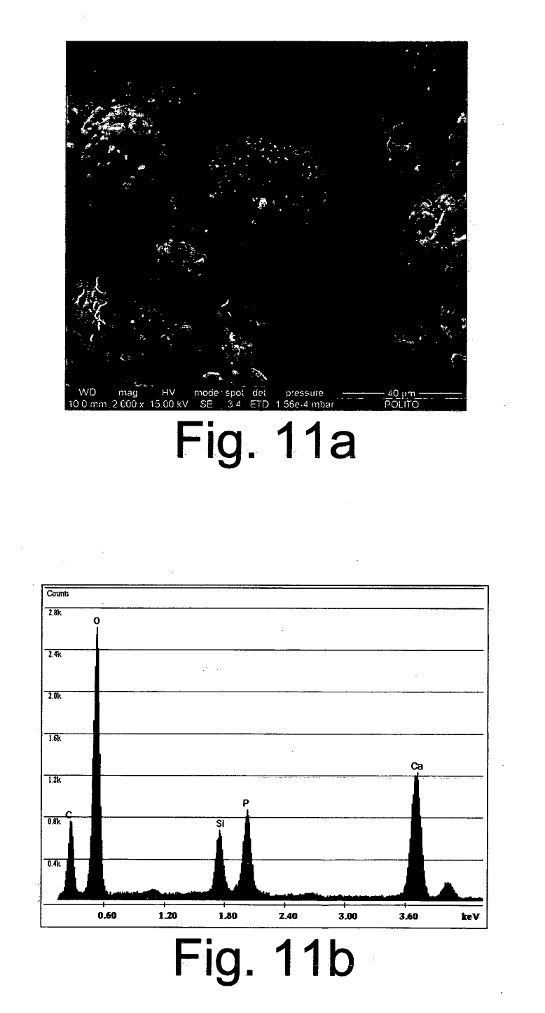

FIG. 11a illustrates a micrograph of the surface of the composite material realized according to example 5 after 1 day of soaking in SBF, in which the formation of many agglomerates of hydroxyapatite micro-crystals is clearly visible both on MBG particles that emerge from the matrix and on the matrix itself;

FIG. 11b reports an EDS composition analysis of the material observed in FIG. 11a;

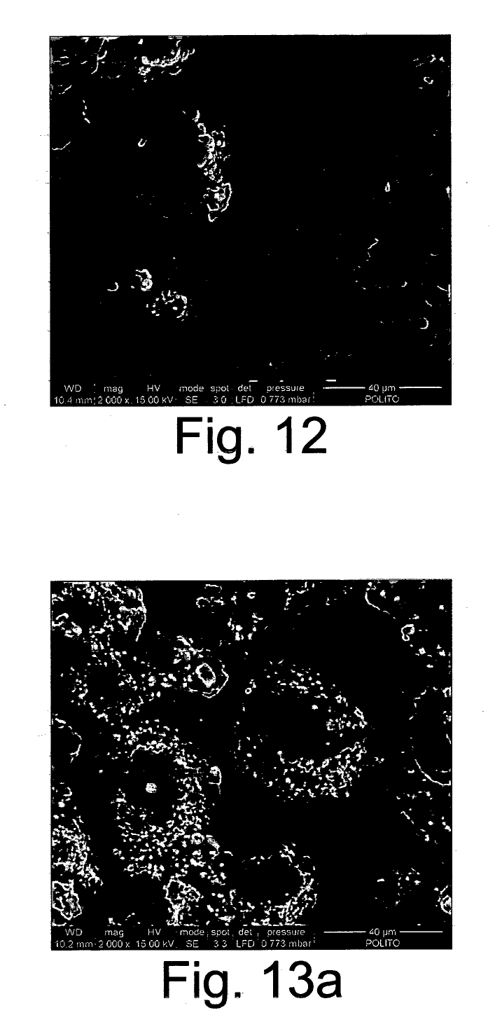

FIG. 12 illustrates a micrograph of the surface of the composite material realized according to example 5 after 3 days of soaking in SBF, which shows an increased amount of hydroxyapatite micro-crystalline agglomerates than that observed in FIG. 11a, consistently with the longer duration of the immersion;

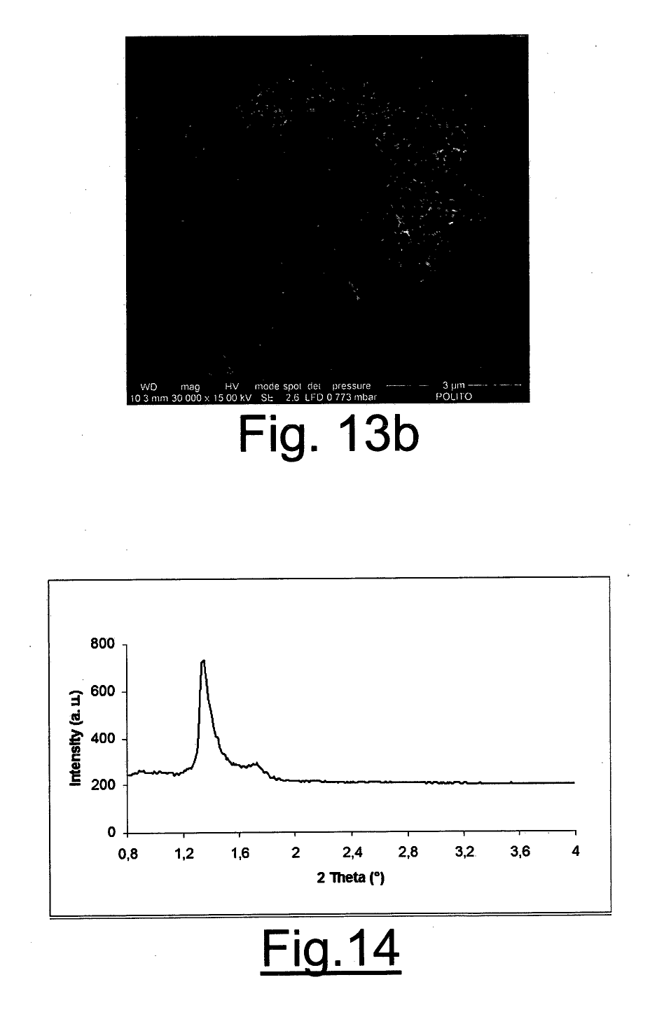

FIG. 13a illustrates a micrograph of the surface of the composite material realized according to example 5 after 7 days of soaking in SBF: as a consequence of the further increase of the test duration compared with the two previous figures, the micro-crystalline agglomerates of hydroxyapatite are homogeneously distributed, so that they form a layer that completely covers both the polymeric matrix and the emerging MBG particles;

FIG. 13b illustrates a micrograph that shows a magnification of the sub-micrometrical hydroxyapatite crystals reported in FIG. 13a, which cover the whole exposed surface of the composite prepared as described in example 5 after 7 days of soaking in SBF;

FIG. 14 reports the spectrum resulted from X-ray diffraction analysis on MBG powders in the range of 2θ angle between 0.6° and 5°: diffraction peaks clearly visible within the angular range 1°-2° demonstrate that nanoporous particles possess a regular structure. For the specific example under consideration, the selected surfactant is Pluronic P123, which determines nanopores characterized by an hexagonal shape with an average pores size of 4.7 nm, as seen in FIG. 19;

FIG. 15 illustrates an X-ray image, obtained through the radiological equipment specific for vertebroplasty, that allows to compare the radio-opacity level of: (a) commercial vertebral cement (CementoFixx® produced by Optimed), that represents the reference material to evaluate the radio-opacity; (b) MBGZ powder, synthesized according to the following example 8; (c) composite cement prepared as described in example 9 below, that is characterized by a calcium sulphate matrix containing MBGZ as one of the dispersed phases. From this figure it is clear that the radio-opacity level conferred by the MBGZ, both as in the form of powder [FT1] and as dispersed phase in an injectable cement [FT2], can be considered more than satisfactory because it is totally comparable, or even higher, to that of vertebral cements available now on the market;

FIG. 16a illustrates a micrograph of the surface of the composite material realized according to example 6 using MBG particles smaller than 32 μm, while FIG. 16b illustrates a micrograph, at the same magnification of FIG. 16a, of the composite material realized according to example 6 using MBG particles whose size ranges within 75 and 106 μm: in both figures polymethylmethacrylate particles, which are spherical, and MBG ones, which are sharp and irregular, are observed. Comparing the two images, it is clearly evident that smaller the size of the particles of glass dispersed phase greater their exposure on the surface: in fact, whereas FIG. 16b shows a clear prevalence of spherical matrix particles among which only a very limited number of MBG particles emerges with a uniform distribution, FIG. 16a exhibits a reversal situation, characterized by an homogeneous layer of dispersed phase among which only few polymethylmethacrylate particles are able to come out. Such unexpected outcomes underline the importance of making an accurate modulation of the particles size and selecting the range that maximizes the exposure of dispersed phase on the surface, in order to optimize its bioactive and osteoinductive function when in contact with body fluids;

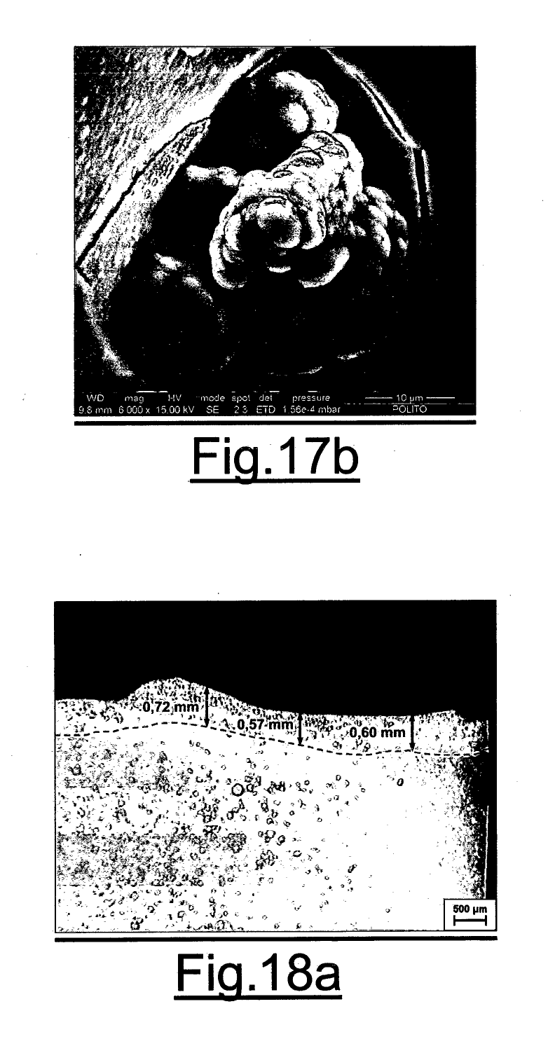

FIG. 17a illustrates a micrograph of a globular hydroxyapatite agglomerate nucleated on MBGZ (prepared in accordance with example 8) after 72 hours of soaking in SBF, demonstrating the excellent bioactivity of MBGZ;

FIG. 17b illustrates a micrograph which shows a layer of hydroxyapatite microcrystals precipitated on MBGZ (prepared in accordance with example 8) after 7 days of soaking in SBF, that represents a further proof of the extremely high bioactivity provided by MBGZ;

FIG. 18a is an image obtained through optical microscope which illustrates the section of a hardened sample of a cement prepared in accordance with example 6, that is a composite constituted by a polymethylmethacrylate matrix containing 20% by weight of MBG powder smaller than 32 μm, as resulted from capillarity test: this was carried out by partially soaking the sample (for half of its height) in a mixture of SBF and a red liquid for a fixed time. From this figure a depth of capillary absorption of about 600 μm is clearly noticeable: this fact demonstrates that, when MBG particles are highly exposed on the surface, as previously shown by FIG. 16a, the presence of their nanopores enables a remarkable capillary absorption of fluids, which is an effect that promotes and increases the bioactivity provided by the osteoinductive behaviour of MBG;

FIG. 18b is an image obtained through optical microscope which illustrates the section of a hardened sample of a composite cement constituted by a polymethylmethacrylate matrix containing 20% by weight of powder smaller than 32 μm of a traditional not-mesoporous bioactive glass (prepared through melting, quenching, milling and sieving), as resulted from capillarity test: this was carried out by partially soaking the sample (for half of its height) in a mixture of SBF and a red liquid for a fixed time. From this figure no layer of fluid absorption can be noticed: therefore, in absence of nanopores almost no capillarity is observed and, by comparison with FIG. 18a, this outcome further confirms the greater technical effect provided by the utilization of MBG with optimized size rather than a traditional bioactive glass;

FIG. 19 reports a curve that describes the size distribution of the nanopores of MBG (used in all examples from 1 to 7), obtained by nitrogen adsorption experiment, calculated using DFT method.

### EXAMPLE 1

Composition Containing:

Matrix Material

α-calcium sulphate hemi-hydrate: 80% by weight

Dispersed Phase

MBG: 20% by weight

MBG Composition:

SiO₂: 80% by moles
CaO: 15% by moles
P₂O₅: 5% by moles
Particle size: below 20 μm
Specific surface (BET): 307 m²/g
Average pore size: 4.7 nm
(calculated using DFT method)

The bone cement composition was obtained by adding to the mixture of the above-mentioned powders an amount of water ranging between 0.1 and 1 ml per gram of the total dry powder and preferably between 0.5 and 0.6 ml/g.

### EXAMPLE 2

Composition Containing:

Matrix Material

α-calcium sulphate hemi-hydrate: 70% by weight

First Dispersed Phase

MBG: 10% by weight

MBG Composition:

SiO₂: 80% by moles
CaO: 15% by moles
P₂O₅: 5% by moles
Particle size: below 20 μm
Specific surface (BET): 307 m²/g
Average pore size: 4.7 nm
(calculated using DFT method)

Second Dispersed Phase

SiO₂—CaO—Na₂O—Al₂O₃ based glass: 20% by weight

Composition of the Second Dispersed Phase:

SiO₂: 57% by moles
CaO: 34% by moles
Na₂O: 6% by moles
Al₂O₃: 3% by moles
Particle size: below 20 μm
Specific surface: a few m²/g
Almost zero porosity
Liquid to powder ratio ranges between 0.5 ml/g and 0.6 ml/g.

### EXAMPLE 3

Composition Containing:

Matrix Material

α-calcium sulphate hemi-hydrate: 70% by weight

First Dispersed Phase

MBG: 10% by weight

MBG Composition:

SiO₂: 80% by moles
CaO: 15% by moles
P₂O₅: 5% by moles
Particle size: below 20 μm
Specific surface (BET): 307 m²/g
Average pore size: 4.7 nm
(calculated using DFT method)

Second Dispersed Phase

20% by weight of SiO₂—CaO—Na₂O—Al₂O₃ based glass-ceramic containing crystals of β-wollastonite Ca(SiO₃):

Composition of the Second Dispersed Phase:

SiO₂: 57% by moles
CaO: 34% by moles
Na₂O: 6% by moles
Al₂O₃: 3% by moles
Particle size: below 20 μm
Specific surface: a few m²/g
Almost zero porosity
Liquid to powder ratio ranges between 0.5 ml/g and 0.6 ml/g.

### EXAMPLE 4

Composition Containing:

Matrix Material

α-calcium sulphate hemi-hydrate: 60% by weight

First Dispersed Phase

MBG: 10% by weight

MBG Composition:

SiO₂: 80% by moles
CaO: 15% by moles
P₂O₅: 5% by moles
Particle size: below 20 μm
Specific surface (BET): 307 m²/g
Average pore size: 4.7 nm
(calculated using DFT method)

Second Dispersed Phase

30% by weight of SiO₂—CaO—Na₂O—Al₂O₃ based glass-ceramic containing crystals of β-wollastonite Ca(SiO₃):

Composition of the Second Dispersed Phase:

SiO₂: 57% by moles
CaO: 34% by moles
Na₂O: 6% by moles
Al₂O₃: 3% by moles
Particle size: below 20 μm
Specific surface: a few m²/g
Almost zero porosity
Liquid to powder ratio ranges between 0.5 ml/g and 0.6 ml/g.

### EXAMPLE 5

Composition Containing:

Matrix Material

polymethylmethacrylate: 70% by weight

Dispersed Phase

MBG: 30% by weight
MBG composition:
SiO₂: 80% by moles
CaO: 15% by moles
P₂O₅: 5% by moles
Particle size: below 20 μm
Specific surface (BET): 307 m²/g
Average pore size: 4.7 nm
(calculated using DFT method)
overall ratio: liquid phase (monomer, catalysts and inhibitors)/solid phase (polymethylmethacrylate, initiator and MBG)=0.5 ml/g
ratio: liquid phase (monomer, catalysts and inhibitors)/(polymethylmethacrylate and initiator)=0.7 ml/g.

### EXAMPLE 6

Composition Containing:

Matrix Material

polymethylmethacrylate: 80% by weight

Dispersed Phase

MBG: 20% by weight

MBG Composition:

SiO₂: 80% by moles
CaO: 15% by moles
P₂O₅: 5% by moles
Particle size: below 32 μm or in the range 75-106 μm
Specific surface (BET): 307 m²/g
Average pore size: 4.7 nm
(calculated using DFT method)
overall ratio: liquid phase (monomer, catalysts and inhibitors)/solid phase (polymethylmethacrylate, initiator and MBG)=0.5 ml/g
ratio: liquid phase (monomer, catalysts and inhibitors)/(polymethylmethacrylate and initiator)=0.6 ml/g.

Mechanical compressive tests according to ISO 5833-2002 standard (that is specific for acrylic resin cements for biomedical applications) were performed on the two compositions object of the present example (among which the only difference is the granulometry of powders of the dispersed phase and which are identified respectively as “PMMA/MBG<32 μm—80/20% wt” and “PMMA/MBG [75; 106]μm—80/20% wt”) in order to determine the values of their compressive strength. For this test, the selected reference is a cement composed of only polymethylmethacrylate matrix (identified as “PMMA—100% wt”), whose samples were tested in the same experimental conditions. The values of compressive strength for the tested cements are reported in the following table in the form required by the standard, that is the strength value in correspondence to both the 2% offset and the upper yield point:

| | R at ε = | R at upper yield |
|---|---|---|
| Tested cement | 2% [MPa] | point [MPa] |
| PMMA 100% wt | 88.4 ± 5.3 | 90.1 ± 5.6 |
| PMMA/MBG<32 μm - 80/20% wt | 97.7 ± 7.5 | 98.1 ± 7.4 |
| PMMA/MBG[75; 106] μm - 80/20% wt | 94.2 ± 2.4 | 94.4 ± 2.4 |

These results suggest that the addition of MBG particles, besides imparting bioactivity to the composite cement, significantly improves its mechanical compressive strength if compared to the reference cement composed of matrix only. Such increase is much more noticeable when smaller dispersed particles are used because they ensure better mixing and dispersion of the different phases, thus providing a greater homogeneity in the properties of the resulting composite material. This observations, besides being a considerable advantage shown by the compositions object of the present invention, represents an unexpected outcome with respect to what is reported in some publications present in the scientific literature, which describe a decrease of the compressive strength when a dispersed phase is added to composite cements compared with simple polymethylmethacrylate, for instance: Rentería-Zamarrón D., Cortés-Hernandez D. A., Bretado-Aragón L., Ortega-Lara W., “Mechanical properties and apatite-forming ability of PMMA bone cements”, Materials and Design, 2009, vol. 30, pag. 3318-3324.

### EXAMPLE 7

Composition Containing:

Matrix Material

polymethylmethacrylate: 90% by weight

Dispersed Phase

MBG: 10% by weight
MBG composition:
SiO₂: 80% by moles
CaO: 15% by moles
P₂O₅: 5% by moles
Particle size: below 20 μm
Specific surface (BET): 307 m²/g
Average pore size: 4.7 nm
(calculated using DFT method)
overall ratio: liquid phase (monomer, catalysts and inhibitors)/solid phase (polymethylmethacrylate, initiator and MBG)=0.4 ml/g
ratio: liquid phase (monomer, catalysts and inhibitors)/(polymethylmethacrylate and initiator)=0.5 ml/g.

### EXAMPLE 8

Here is described an example of synthesis of a bioactive mesoporous glass containing zirconia as radio-opaque agent, identified as MBGZ, having the following composition:

SiO₂: 73% by moles
CaO: 15% by moles
P₂O₅: 5% by moles
ZrO₂: 7% by moles

MBGZ was synthesized by using the commercial non-ionic block copolymer Pluronic P123 (EO₂₀PO₇₀EO₂₀, where “EO” is poly(ethylene glycol) and “PO” is polypropylene glycol)) as organic surfactant, which acts as structure-directing agent for pores formation.

In a typical synthesis of MBGZ, a synthesis batch is prepared by dissolving P123 (4 g), tetraethylorthosilicate (TEOS, 6.10 g), calcium nitrate tetrahydrate (Ca(NO₃)₂.4H₂O, 1.42 g), triethylphosphate (TEP, 0.73 g), zirconium propoxide (0.92 g), acetylacetone (Acac, which acts as a stabilizer to prevent the zirconium propoxide from uncontrollable hydrolysis and consequent precipitation, 0.10 g) and HCl 0.5 M (1 g) in ethanol (60 g). This synthesis batch is continuously stirred at 35° C. for 24 hours. The resulting sol is cast into Petri dishes to undergo an ageing phase (24 hours at room temperature and 24 hours at 120° C.), during which the evaporation-induced self-assembly (EISA) process occurs. The dried gel is, finally, calcined at 750° C. for 5 hours to obtain the final MBGZ product.

In order to obtain MBGZ powder to be used as dispersed phase in the composite cements object of the present invention, MBGZ is ground and sieved to select particles of the desired size.

### EXAMPLE 9

Composition Containing:

Matrix Material

α-calcium sulphate hemi-hydrate: 70% by weight

First Dispersed Phase

MBGZ: 10% by weight

MBGZ Composition:

SiO₂: 73% by moles
CaO: 15% by moles
P₂O₅: 5% by moles
ZrO₂: 7% by moles
synthesized as described in example 8.
Particle size: below 20 μm
Specific surface (BET): 320 m²/g
Average pore size: 4.4 nm
(calculated using DFT method)

Second Dispersed Phase

20% by weight of SiO₂—CaO—Na₂O—Al₂O₃ based glass-ceramic containing crystals of β-wollastonite Ca(SiO₃):

Composition of the Second Dispersed Phase:

SiO₂: 57% by moles
CaO: 34% by moles
Na₂O: 6% by moles
Al₂O₃: 3% by moles
Particle size: below 20 μm
Specific surface: a few m²/g
Almost zero porosity
Liquid to powder ratio ranges between 0.5 ml/g and 0.6 ml/g.

1-15. (canceled)

16. An injectable composition for bone filling or consolidation, for use in surgery or therapy, comprising a biocompatible material in form of powder, which is hardenable or cross-linkable and selected from calcium sulphate hemi-hydrate and polymethylmethacrylate, a hardening or cross-linking agent for said material and a porous silica-based material in form of powder, having a specific surface area higher than 100 m²/g, which comprises a mesoporous bioactive glass, obtainable by a sol-gel method with the addition of a surfactant as structure-directing agent for pores formation, comprising an oxide mixture containing:
SiO₂: from 60 to 85% by moles
CaO: from 10 to 30% by moles
P₂O₅: from 2 to 10% by moles
said composition, when applied to an affected area of a patient, being adapted to form a composite material wherein said hardenable or cross-linkable material constitutes a matrix and said mesoporous bioactive glass constitutes a dispersed phase in said matrix.

17. A composition according to claim 16, comprising a radio-opaque mesoporous bioactive glass comprising ZrO₂ or SrO or mixtures thereof in an amount from 2 to 15% by moles referred to 100 moles of the mixture of SiO₂, CaO and P₂O₅ oxides.

18. A composition according to claim 16, wherein said mesoporous bioactive glass is a powder with particles size ranging between 5 and 32 μm as obtainable through sieving.

19. A composition according to claim 16, comprising a further dispersed phase, of glassy nature, in the form of a powder or fibres based on SiO₂ and CaO, with the optional addition of one or more oxides selected from the group consisting of Na₂O, MgO, K₂O, Al₂O₃, ZrO₂, SrO and TiO₂ and mixtures thereof.

20. A composition according to claim 16, further comprising an additional bioresorbable dispersed phase in the form of powders or fibres, based on P₂O₅ and CaO, containing one or more oxides selected from the group consisting of Na₂O, MgO, K₂O and TiO₂ and mixtures thereof.

21. A composition according to claim 16, wherein said matrix material comprises powders of α-calcium sulphate hemi-hydrate and in that it further comprises an agent delaying the onset of the hardening, such as carboxylic acids selected from tartaric acid, citric acid, succinic acid, malic acid and mixtures thereof.

22. A composition according to claim 21, comprising as matrix material a calcium phosphate or calcium phosphates, in combination with calcium sulphate hemi-hydrate.

23. A composition according to claim 16, comprising from 40% to 95% by weight, preferably from 60% to 90% by weight of matrix material, referred to 100% by weight of the matrix material and dispersed phases.

24. A composition according to claim 16, comprising an aqueous carrier selected from distilled water, balanced saline solution, optionally containing a radio-opaque agent.

25. A composition according to claim 24, further comprising a biocompatible organic oil.

26. A composition according to claim 16, wherein said mesoporous bioactive glass is loaded with biologically active substances, selected from antibiotic drugs, anti-inflammatories, painkillers, anti-tumorals and growth factors, particularly morphogenetic proteins.

27. A composition according to claim 16, wherein said mesoporous bioactive glass has a specific surface area comprised between 300 m²/g and 500 m²/g and a pore dimension/size comprised between 2 nm and 10 nm.

28. A composite bone cement obtained from a composition according to claim 16.

29. A radio-opaque mesoporous bioactive glass comprising an oxide mixture containing:
SiO₂: from 60 to 85% by moles
CaO: from 10 to 30% by moles
P₂O₅: from 2 to 10% by moles, and
with reference to 100 moles of the above-mentioned oxides, a quantity of an oxide selected from SrO and ZrO₂ and mixtures thereof that is sufficient to confer radio-opacity to said glass.

30. A method of preparing bone cements comprising calcium sulphate hemi-hydrate or polymethylmethacrylate comprising adding to said calcium sulphate hemi-hydrate or polymethylmethacrylate a mesoporous bioactive glass according to claim 29 as a dispersed phase.
